SEQUENCE LISTING (11 sequences)

SEQ ID NO: 1 - DNA, 33 bp, Artificial Sequence
  Synthetic
ttagaccgaaacaatgtggaattggccaataca

SEQ ID NO: 2 - DNA, 16 bp, Artificial Sequence
  Synthetic
tgtattggccaattcc

SEQ ID NO: 3 - DNA, 32 bp, Artificial Sequence
  Synthetic
acattgtttcggtctaacgagtacagtatcag

SEQ ID NO: 4 - DNA, 48 bp, Artificial Sequence
  Synthetic
gctcatgtcatagtctacgatgctcaggctgtgcgagtacagtatcag

SEQ ID NO: 5 - DNA, 48 bp, Artificial Sequence
  Synthetic
gactatgacatgagctacgatgctcaggctgtgctgatactgtactcg

SEQ ID NO: 6 - DNA, 17 bp, Artificial Sequence
  Synthetic
agccctgacgcatcgta

SEQ ID NO: 7 - DNA, 12 bp, Artificial Sequence
  Synthetic
gatgctcaggct

SEQ ID NO: 8 - DNA, 54 bp, Artificial Sequence
  Synthetic
gactatgacgtcatagtctacgatgctcaggctgtgctgatactgcagtatcag

SEQ ID NO: 9 - DNA, 24 bp, Artificial Sequence
  Synthetic
agtctaggattcggcgtgggttaa

SEQ ID NO: 10 - DNA, 60 bp, Artificial Sequence
  Synthetic
ttaacccacgccgaatcctagacttacgatgctcaggctgtgagtctaggattcggcgtg

SEQ ID NO: 11 - DNA, 60 bp, Artificial Sequence
  Synthetic
agtctaggattcggcgtgtacgatgctcaggctgtgcacgccgaatcctagactactttg

(19) United States
(12) Patent Application Publication
(10) Pub. No.: US 2018/0127803 A1
(43) Pub. Date: May 10, 2018

(54) ULTRA SENSITIVE PROBES FOR DETECTION OF NUCLEIC ACID

(71) Applicant: QuanDx Inc., San Jose, CA (US)

(72) Inventors: Xiaojun LEI, San Jose, CA (US); Yuan YUAN, San Jose, CA (US); Qiang LI, San Jose, CA (US); Yi ZHANG, Wilmette, IL (US)

(21) Appl. No.: 15/573,131

(22) PCT Filed: May 10, 2016
(86) PCT No.: PCT/US16/31552

§ 371 Date: Nov. 10, 2017

Related U.S. Application Data
(60) Provisional application No. 62/159,318, filed on May 10, 2015.

(51) Int. Cl. C12Q 1/682; C12Q 1/70

(52) U.S. Cl. CPC C12Q 1/682; C12Q 2563/149; C12Q 2525/161; C12Q 1/70

(57) ABSTRACT

The present disclosure provides a composition with ultra sensitivity for detection of nucleic acid and the method of use thereof. The composition comprises a target probe capable of hybridizing to a target nucleic acid, at least one first bridge probe, at least one second bridge probe, and a label probe. The target probe includes a pre-bridge region having a first tail nucleotide sequence. The first bridge probe includes sequentially a first head region having a first head nucleotide sequence, a first gap region having a gap nucleotide sequence, and a first tail region having the first tail nucleotide sequence. The second bridge probe includes sequentially a second head region having a second head nucleotide sequence complementary to the first head nucleotide sequence, a second gap region having the gap nucleotide sequence, and a second tail region having a second tail nucleotide sequence complementary to the first tail nucleotide sequence. The label probe is capable of hybridizing to the first and the second gap nucleotide region.

### CROSS-REFERENCE TO RELATED APPLICATIONS

This application claims priority to U.S. provisional patent application No. 62/159,318, filed May 10, 2015, the disclosure of which is incorporated herein by reference.

### FIELD OF THE INVENTION

The present invention generally relates to nucleic acid chemistry and assays. More particularly, the invention relates to probes and methods for detection of nucleic acid in a sample.

### BACKGROUND OF THE INVENTION

Hybridization-based methods for detection of nucleic acid, such as Northern and Southern blot, in situ hybridization, have broad application in molecular diagnostic and biomedical research. In principle, a hybridization probe is generated by conjugating a label that provides detectable signal, such as radioactivity and fluorescence, to a fragment of DNA or RNA with sequence complementary to a target sequence. The hybridization probe hybridizes to single-stranded nucleic acid (DNA or RNA) containing the target sequence due to complementarity between the probe and target. The signal from the label is detected to determine the presence or absence of the target sequence.

However, the application of hybridization probe can be limited by its inability to detect DNA or RNA targets with low copy numbers due to lack of sensitivity and specificity. Therefore, there is continuing need to develop probes with ultra sensitivity to detect nucleic acids.

### BRIEF SUMMARY OF THE INVENTION

In one aspect, the present disclosure provides a composition comprising a target probe capable of hybridizing to a target nucleic acid, at least one first bridge probe, at least one second bridge probe, and at least one label probe having a label. The target probe includes a pre-bridge region having a first tail nucleotide sequence. The first bridge probe includes sequentially a first head region having a first head nucleotide sequence, a first gap region having a gap nucleotide sequence, and a first tail region having the first tail nucleotide sequence. The second bridge probe includes sequentially a second head region having a second head nucleotide sequence complementary to the first head nucleotide sequence, a second gap region having the gap nucleotide sequence, and a second tail region having a second tail nucleotide sequence complementary to the first tail nucleotide sequence. The label probe is capable of hybridizing to the first and the second gap nucleotide region.

In certain embodiments, the first head nucleotide sequence consists of 4-20 nucleotides (e.g., 3, 4, 5, 6, 7, 8, 9, 10, 11, 12, 13, 14, 15, 16, 17, 18, 19 or 20 nucleotides).

In certain embodiments, the first tail nucleotide sequence consists of 4-20 nucleotides (e.g., 3, 4, 5, 6, 7, 8, 9, 10, 11, 12, 13, 14, 15, 16, 17, 18, 19 or 20 nucleotides).

In certain embodiments, the gap nucleotide sequence consists of 6-20 nucleotides (e.g., 6, 7, 8, 9, 10, 11, 12, 13, 14, 15, 16, 17, 18, 19 or 20 nucleotides).

In certain embodiments, the label is a fluorophore, a horse radish peroxidase or an alkaline phosphatase.

In certain embodiments, the probe (e.g., the target probe, bridge probe, label probe) described herein can comprise one or more nucleotide analogs (e.g., altered backbone, sugar, or nucleobase). In certain embodiments, the nucleotide analog is selected from the group consisting of 5-bromouracil, a peptide nucleic acid nucleotide, a xeno nucleic acid nucleotide, a morpholino, a locked nucleic acid nucleotide, a glycol nucleic acid nucleotide, a threose nucleic acid nucleotide, a dideoxynucleotide, a cordycepin, a 7-deaza-GTP, a fluorophore (e.g. rhodamine or flurescein linked to the sugar), a thiol containing nucleotide, a biotin linked nucleotide, a fluorescent base analog, a methyl-7-guanosine, a methylated nucleotide, an inosine, thiouridine, a pseudourdine, a dihydrouridine, a queuosine, and a wyosine. In certain embodiments, the nucleotide analog is a locked nucleic acid nucleotide.

In certain embodiments, the target nucleic acid is selected from the group consisting of a DNA, a cDNA, a RNA, a mRNA, a rRNA, a miRNA, a Lnc RNA and a siRNA.

In certain embodiments, the target probe further comprises a label region having the gap nucleotide sequence.

In certain embodiments, the composition described above further comprises a capture probe capable of hybridizing to the target nucleic acid, said capture probe having a magnetic bead or a biotin.

In one embodiment, the first bridge probe is the same as the second bridge probe.

In one embodiment, the present disclosure provides a composition comprising a target probe capable of hybridizing to a target nucleic acid, at least one bridge probe and a label probe capable of hybridizing to the bridge probe. The target probe comprises a pre-bridge region having a tail palindromic sequence. The bridge probe comprises sequentially (i) a first region having a head palindromic sequence; (ii) a second region having a gap sequence; and (iii) a third region having the tail palindromic sequence. The label probe comprises a label.

In certain embodiments, the head palindromic sequence consists of 4-20 nucleotides (e.g., 3, 4, 5, 6, 7, 8, 9, 10, 11, 12, 13, 14, 15, 16, 17, 18, 19 or 20 nucleotides).

In certain embodiments, the tail palindromic sequence consists of 4-20 nucleotides (e.g., 3, 4, 5, 6, 7, 8, 9, 10, 11, 12, 13, 14, 15, 16, 17, 18, 19 or 20 nucleotides).

In another aspect, the present disclosure provides a method of detecting a target nucleic acid in a sample. According to one embodiment, the method comprises the steps of: a) contacting a target probe to the target nucleic acid; b) associating a first bridge probe, a second bridge probe and a label probe with the target probe; and c) detecting the presence, absence, or amount of the label probe associated with the target molecule. The target probe is capable of hybridizing to the target nucleic acid and comprises a pre-bridge region having a tail nucleotide sequence. The first bridge probe comprises sequentially (i) a first head region having a first head nucleotide sequence; (ii) a first gap region having a gap nucleotide sequence; and (iii) a first tail region having the tail nucleotide sequence. The second bridge probe comprises sequentially (i) a second head region having a second head nucleotide sequence complementary to the first head nucleotide sequence; (ii) a second gap region having the gap nucleotide sequence; (iii) a second tail region having a second tail nucleotide sequence complementary to the first tail nucleotide sequence. The label probe is capable of hybridizing to the bridge probe and has a label.

In certain embodiments, the sample is selected from the group consisting of sera, plasma, saliva, urine, cell and tissue.

In certain embodiments, the method further comprises the step of capturing said target nucleic acid with a capture probe comprising a magnetic bead or a biotin, wherein the capture probe is capable of hybridizing to the target nucleic acid.

### BRIEF DESCRIPTION OF THE FIGURES

FIG. 1 shows an ultra sensitive probe composition according to an embodiment of the invention.

FIG. 2 shows an ultra sensitive probe composition according to another embodiment of the invention.

FIG. 3 shows the sequences of the target nucleic acid (Target), the capture probe (Biotin-I), the target probe (II), the first bridge probe (O1), the second bridge probe (O2) and label probe (Yin-Yang Probe P1) used in Example 1.

FIG. 4 shows a method of detecting a target nucleic acid using an ultra sensitive probe composition according Example 1.

FIG. 5A shows the detection of the target nucleic acid using the probe composition of Example 1.

FIG. 5B shows that both the first bridge probe and the second bridge probe are required for the detection of the target nucleic acid in Example 1.

FIG. 6 shows the sequence of bridge probe (O3) and using O3 to detect the target nucleic acid of Example 2.

FIG. 7 shows a method of detecting a target nucleic acid using a probe composition according to Example 3.

FIG. 8 shows the sequences of the target nucleic acid, the first bridge probe (H1), and the second bridge probe (H2) used in Example 3.

FIG. 9 shows the principle of detecting the target nucleic acid in Example 3.

FIG. 10 shows the detection of the target nucleic acid using the probe composition of Example 3.

### DETAILED DESCRIPTION OF THE INVENTION

In the Summary of the Invention above and in the Detailed Description of the Invention, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or particular claim, that feature can also be used, to the extent possible, in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, the embodiments described herein can be practiced without there specific details. In other instances, methods, procedures and components have not been described in detail so as not to obscure the related relevant function being described. Also, the description is not to be considered as limiting the scope of the implementations described herein. It will be understood that descriptions and characterizations of the embodiments set forth in this disclosure are not to be considered as mutually exclusive, unless otherwise noted.

The following definitions are used in the disclosure:

It is understood that as used herein and in the appended claims, the singular forms “a,” “an,” and “the” include the plural reference unless the context clearly dictates otherwise. Thus, for example, a reference to a “bridge probe” is a reference to one or more bridge probes, and includes equivalents thereof known to those skilled in the art and so forth.

As used herein, “associate” or “associating” means physically direct or indirect attachment. For example, the label probe can hybridize to one or more bridge probe, which hybridizes to the target probe, which hybridizes the target nucleic acid, thereby the label probe is associated with the target nucleic acid.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 4 to 20 nucleotides means a range whose lower limit is 4 nucleotides, and whose upper limit is 20 nucleotides.

A “bridge probe” as used herein is a nucleic acid that is capable of hybridizing to the target probe and the label probe. The bridge probe is also capable of hybridizing with other bridge probe and forming a ladder-shaped structure, which is capable of hybridizing to multiple label probes, thus amplifying the signal detectable. Typically, the bridge probe includes sequentially a first region having a head nucleotide sequence, a gap region having a gap nucleotide sequence, and a tail region having a tail nucleotide sequence. In certain embodiments, the bridge probe includes sequentially a first palindromic nucleotide sequence (i.e., the head palindromic sequence), a second nucleotide sequence (i.e., the gap nucleotide sequence) that is complementary to a nucleotide sequence of the label probe; and a third palindromic nucleotide sequence (i.e., the tail palindromic sequence). For example, a bridge probe can have a head palindromic sequence 5′-AGCT-3′ and a tail palindromic sequence 5′-GCGC-3′.

As used herein, a “capture probe” refers to a polynucleotide that is capable of hybridizing to the target nucleic acid. Typically, the capture probe includes a nucleotide sequence that is complementary to a sequence of the target nucleic acid. In preferred embodiments, the sequence of the target nucleic acid that is complementary to the capture probe is not overlapping with the sequence complementary to the target probe. In certain embodiments the capture probe is conjugated to a magnetic bead, through which a complex comprising the target nucleic acid and the ultra sensitive composition disclosed herein can be captured.

As used herein, the term “nucleic acid” (interchangeable with the term “polynucleotide”) encompasses any physical string of monomer units that can be corresponded to any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not conventional to biological RNA or DNA, such as 2′-O-methylated oligonucleotides), and the like. The nucleotides of the polynucleotide can be deoxyribonucleotides, ribonucleotides or nucleotide analogs, can be natural and unnatural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

As used herein, a “nucleotide analog” refers to a nucleotide (deoxyribonucleotide or ribonucleotide) comprising one or more modifications (e.g. altered backbone, sugar, or nucleobase). Some non-limiting examples of nucleotide analogs include: 5-bromouracil, peptide nucleic acid nucleotides, xeno nucleic acid nucleotides, morpholinos, locked nucleic acid nucleotides, glycol nucleic acid nucleotides, threose nucleic acid nucleotides, dideoxynucleotides, cordycepin, 7-deaza-GTP, florophores (e.g., rhodamine or flurescein linked to the sugar), thiol containing nucleotides, biotin linked nucleotides, fluorescent base analogs, CpG islands, methyl-7-guanosines, methylated nucleotides, inosines, thiouridines, pseudourdines, dihydrouridines, queuosines, and wyosines.

Xeno nucleic acid (XNA) refers to a group of synthetic polymers similar to DNA and RNA that differ in the sugar backbone. Examples of XNA include without limitation 1,5-anhydrohexitol nucleic acid (HNA), cyclohexene nucleic acid (CeNA), Threose nucleic acid (TNA), glycol nucleic acid (GNA), locked nucleic acid (LNA), peptide nucleic acid (PNA).

Peptide nucleic acid (PNA) is an artificial synthesized polymer similar to DNA or RNA. While DNA and RNA have a deoxyribose and ribose sugar backbone, respectively, PNA's backbone is composed of repeating N-(2′aminoethyl)-glycine units linked by peptide bonds. The various purine and pyrimidine bases are linked to the backbone by a methylene bridge (—CH₂—) and a carbonyl group (—(C═O)—). Because the backbone of PNA contains no charged phosphate groups, the binding between PNA/DNA strands is stronger than between DNA/DNA strands due to the lack of electrostatic repulsion, resulting in increased melting temperature.

A locked nucleic acid is a modified RNA nucleotide whose ribose moiety is modified with an extra bridge connecting the 2′ oxygen and 4′ carbon. The bridge “locks” the ribose in the 3′-endo conformation, which enhances base stacking and backbone pre-organization of the locked nucleic acid, thus significantly increases its hybridization properties (melting temperature).

Threose nucleic acid (TNA) has a backbone structure composed of repeating threose sugars linked together by phosphodiester bonds. TNA can self-assemble by Wastson-Crick base pairing into duplex structure and can form base pairs complementary to strands of DNA and RNA.

Glycol nucleic acid (GNA) has a backbone composed of repeating glycol units linked by phosphodiester bonds. GNA shows a stronger Watson-Crick base pairing than DNA and RNA and requires a high temperature to melt a duplex GNA or GNA/DNA, GNA/RNA.

A “nucleic acid target” or “target nucleic acid” means a nucleic acid, or optionally a region thereof, that is to be detected.

As used herein, a “nucleotide sequence” or “polynucleotide sequence” is a polymer of nucleotides (an oligonucleotide, a DNA, a nucleic acid, etc.) or a character string representing a nucleotide polymer, depending on context. From any specified nucleotide sequence, either the given nucleic acid or the complementary nucleic acid sequence can be determined.

A “label” as used herein is a moiety that facilitates detection of a molecule, typically by directly or indirectly providing a detectable signal. Common labels in the context of the present invention include fluorescent, luminescent, light-scattering, and/or colorimetric labels. Suitable labels include fluorophore and enzymes, such as Horse Radish Peroxidase (HRP), Alkaline Phosphatase (AP), as well as radionuclides, substrates, cofactors, inhibitors, chemiluminescent moieties, magnetic particles, and the like.

As used herein, a “label probe” refers to an entity that binds to a target molecule, directly or indirectly, and enables the target molecule to be detected, e.g., by a readout instrument. A label probe is typically a single-stranded polynucleotide that comprises one or more label that directed or indirectly provides a detectable signal. In certain embodiments, a label probe is a double-stranded polynucleotide comprising a label capable of providing detectable signal. The label can be covalently linked to the polynucleotide, or the polynucleotide can be configured to bind to the label (e.g., a biotinylated polynucleotide can bind a streptavidin associated label). The label probe can, for example, hybridize directly to a target nucleic acid, or it can hybridize to a nucleic acid (e.g., a target probe) that is in turn hybridized to the target nucleic acid or to one or more other nucleic acids that are hybridized to the nucleic acid. In preferred embodiments, the label probe can comprise a nucleotide sequence that is complementary to a nucleotide sequence (e.g., a gap nucleotide sequence) in a target probe, a bridge probe, or the like.

A “palindromic sequence” is a nucleotide sequence that is the same whether read 5′ to 3′ on one strand or 5′ to 3′ on the complementary strand with which it forms a double helix. For example, the DNA sequence 5′-AGCT-3′ is palindromic because its nucleotide-by-nucleotide complement is 3′-TCGA-5′, which gives the original sequence from 5′ to 3′.

As used herein, a “probe” is an entity that can be used in the detection of a target molecule. Typically, a probe in the present disclosure refers to a nucleic acid molecule, with or without modification.

The term “sample” as used herein refers to any sample having or suspect of having the target nucleic acid, including sample of biological tissue or fluid origin, obtained, reached, or collected in vivo or in situ. Exemplary biological samples include but are not limited to cell lysate, a cell culture, a cell line, a tissue, an organ, a biological fluid, and the like. In certain embodiments, the sample is selected from the group consisting of sera, plasma, saliva, urine, cell and tissue.

The term “sequentially” means that the components (e.g., the head palindromic sequence, the gap sequence, and the tail palindromic sequence) in the bridge probe are juxtaposed in a 5′ end to 3′ end, or 3′ to 5′ order. For one example, the head palindromic sequence is located at the 5′end of the bridge probe, the gap sequence is located in the downstream of the head palindromic sequence, and the tail palindromic sequence is located at the 3′ end of the bridge probe. It is understood that the bridge probe may include additional nucleotide sequence in adjacent to each component (e.g., the head palindromic sequence, the gap sequence, and the tail palindromic sequence) or between two components that does not interfere with the function of the bridge probe.

As used herein, a “target probe” refers to polynucleotide that is capable of hybridizing to a target nucleic acid and associating a label probe with the target nucleic acid. The target probe can hybridize to one or more nucleic acids (i.e., a bridge probe) that in turn hybridize to the label probe, or, in certain embodiments, it can hybridize directly to the label probe. The target probe thus includes a first nucleotide sequence that is complementary to a nucleotide sequence of the target nucleic acid and a second nucleotide sequence (i.e., tail palindromic sequence) that is complementary to a nucleotide sequence (i.e., tail palindromic sequence) of the bridge probe. In certain embodiments, the target probe further includes a third nucleotide sequence (e.g., the gap nucleotide sequence) that is complementary to a nucleotide sequence of the label probe. The target probe is preferably single-stranded.

### Ultra Sensitive Probe Composition

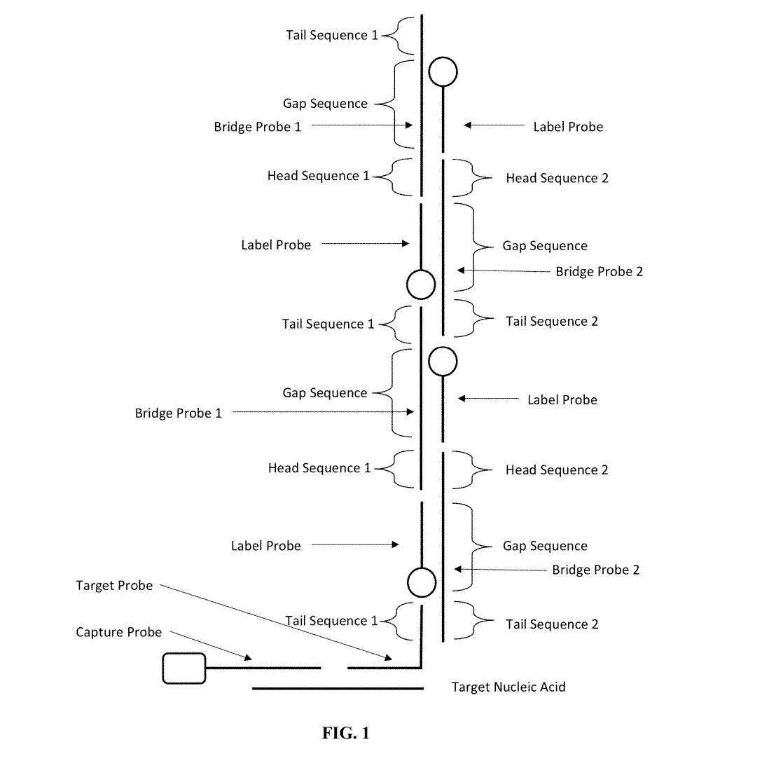

In one aspect, the present disclosure provides a probe composition with ultra sensitivity for detection of nucleic acid. An exemplary embodiment of ultra sensitive probe composition described herein is illustrated in FIG. 1. Referring to FIG. 1, the probe composition is composed of Target Probe, at least one Bridge Probe 1 (two are shown), at least one Bridge Probe 2 (two are shown), and Label Probes (four are shown). Target Probe includes sequentially (e.g., from 5′ to 3′) a target region of a nucleotide sequence complementary to a region of Target Nucleic Acid and a pre-bridge region having a nucleotide sequence of Tail Sequence 1. Bridge Probe 1 includes sequentially (e.g., from 5′ to 3′) a first head region having a nucleotide sequence of Head Sequence 1, a gap region having a nucleotide sequence of Gap Sequence, and a tail region having a nucleotide sequence of Tail Sequence 1. Bridge Probe 2 includes sequentially (e.g., from 5′ to 3′) a head region having a nucleotide sequence of Head Sequence 2, which is complementary to Head Sequence 1, a gap region having a nucleotide sequence of Gap Sequence, and a tail region having a nucleotide sequence of Tail Sequence 2, which is complementary to Tail Sequence 1. Label Probe includes a region having a nucleotide sequence complementary to Gap Sequence, and is capable of hybridizing to the gap region.

When Target Probe, Bridge Probe 1, Bridge Probe 2 and Label Probes are present in suitable solution and at suitable temperature (e.g., about 4° C. to about 50° C.), a complex is simultaneously formed that can be used to detect Target Nucleic Acid. Referring to FIG. 1, Target Probe hybridizes to Target Nucleic Acid through the target region. At the same time, Target Probe hybridizes to Bridge Probe 2 through the hybridization of Tail Sequence 1 and Tail Sequence 2. Bridge Probe 2 further hybridizes to Bridge Probe 1 through hybridization of Head Sequence 1 and Head Sequence 2 and hybridizes to Label Probe at the gap region. Bridge Probe 1 then further hybridizes with another Bridge Probe 2 through hybridization of Tail Sequence 1 and Tail Sequence 2, and also hybridizes to Label Probe at the gap region. As such, a probe complex can be formed through the hybridization chain reaction in which each bridge probe hybridizes to two other bridge probes at its head and tail region, respectively, and a label probe at the gap region. The probe complex can include numerous label probes that generate signal strong enough to be able to detect target nucleic acid of low amount.

In certain embodiments, the ultra sensitive probe composition further includes a capture probe. The capture probe is capable to hybridize to the target nucleic acid and includes a reagent that can be used to isolate, enrich or purify the target nucleic acid coupled to the ultra sensitive probe composition. In one embodiment, the capture probe is operably linked to a magnetic bead. In another embodiment, the capture probe is operably linked to a biotin, which can interact with a streptavidin-coupled bead.

In certain embodiments, a Yin-Yang probe (see U.S. Pat. No. 7,799,522, which is incorporated herein through reference) can be used as the label probe. A Yin-Yang probe is a double-stranded probe made of two complementary oligonucleotides of different lengths. One strand is labeled with a fluorophore and the other strand is labeled with a quencher. When self-hybridized in a stable double-stranded structure, the fluorophore and the quencher are in close proximity, thus the fluorophore is quenched by the quencher and the probe is non-fluorescent. In the presence of the target nucleic acid, the longer strand of the probe can spontaneously bind to the target nucleic acid, the double-stranded probe becomes dissociated, and the fluorophore become fluorescent. Using Yin-Yang probe can decrease background signal and further increase the sensitivity of the ultra sensitive composition disclosed herein.

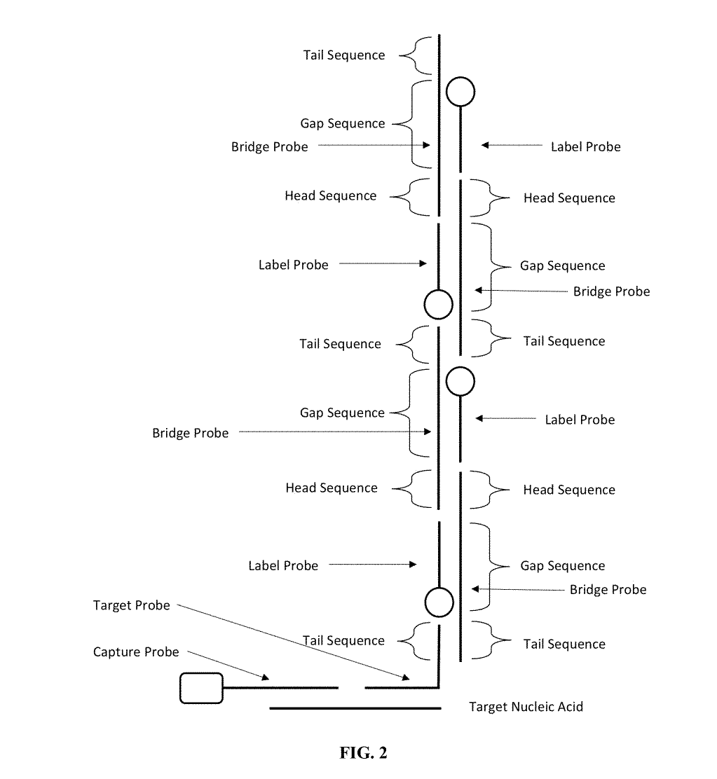

In certain embodiments, Bridge Probe 1 and Bridge 2 as illustrated in FIG. 1 can be the same. FIG. 2 illustrates one of such embodiments. Referring to FIG. 2, the probe composition is composed of Target Probe, at least one Bridge Probe (four are shown), and Label Probes (four are shown). Target Probe includes sequentially (e.g., from 5′ to 3′) a target region of a nucleotide sequence complementary to a region of Target Nucleic Acid and a pre-bridge region having a nucleotide sequence of Tail Sequence. Bridge Probe includes sequentially (e.g., from 5′ to 3′) a first head region having a nucleotide sequence of Head Sequence, a gap region having a nucleotide sequence of Gap Sequence, and a tail region having a nucleotide sequence of Tail Sequence. Each of Head Sequence and Tail Sequence is independently a palindromic sequence. As a result, Head Sequence can hybridize to another Head Sequence. Likewise, Tail Sequence can hybridize to another Tail Sequence. Label Probe includes a region having a nucleotide sequence complementary to Gap Sequence, and is capable of hybridizing to the gap region.

When Target Probe, Bridge Probe and Label Probes are present in suitable solution and at suitable temperature (e.g., about 4° C. to about 50° C.), a complex is simultaneously formed that can be used to detect Target Nucleic Acid. Referring to FIG. 2, Target Probe hybridizes to Target Nucleic Acid through the target region. At the same time, Target Probe hybridizes to Bridge Probe through the hybridization of two Tail Sequences, one on Target Probe, another on Bridge Probe. Bridge Probe further hybridizes to another Bridge Probe through hybridization of Head Sequences and also hybridizes to Label Probe at the gap region. As such, a probe complex can be formed through the chain reaction in which each bridge probe hybridizes to two other bridge probes at its head and tail region, respectively, and a label probe at the gap region. The probe complex can include numerous label probes that generate signal strong enough to be able to detect target nucleic acid of low amount.

The following examples are presented to illustrate the present invention. They are not intended to limiting in any manner.

### Example 1

This example illustrates the detection of a target nucleic acid using a probe composition includes a target probe, a capture probe, a first bridge probe, a second bridge probe and a label probe.

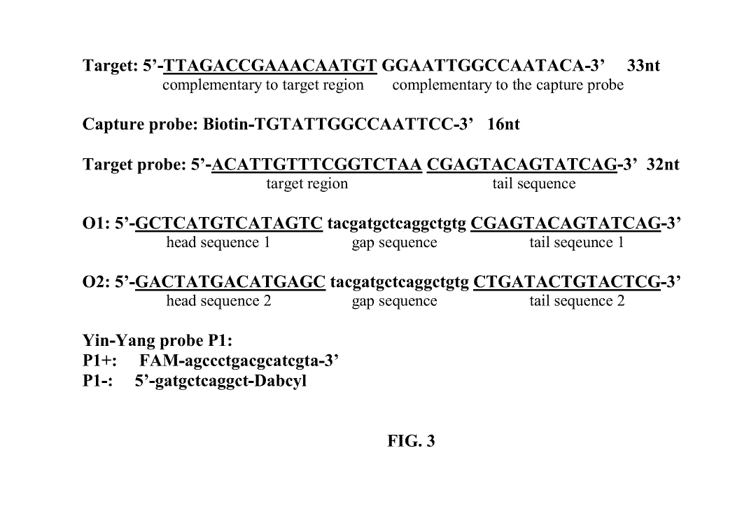

FIG. 3 shows the nucleotide sequence of the target nucleic acid (Target), the capture probe (Biotin-I), the target probe (II), the first bridge probe (01), the second bridge probe (02) and the label probe (P1).

The process of the detection method is illustrated in FIG. 3. Referring to FIG. 3, the target nucleic acid was mixed with the capture probe (Biotin-I), the target probe (II), the first bridge probe (01), the second bridge probe (02) and the label probe (P1 or P1+) in a solution of 10 mM Tris, 15 mM MgCl₂. The target nucleic acid, the capture probe, the target probe, the first bridge probes, the second bridge probes and the label probes then formed a complex, which was pulled down by streptavidin coated magnetic beads (Nvigen Cat # K61002). The complex was then eluted from the Streptavidin coated magnetic beads by water/or a solution of 10 mM Tris, 15 mM MgCl₂, 95 degree boil for 5 min. The fluorescent signals from the eluted complex was then determined using a fluorescence reader (BioRad CFX96).

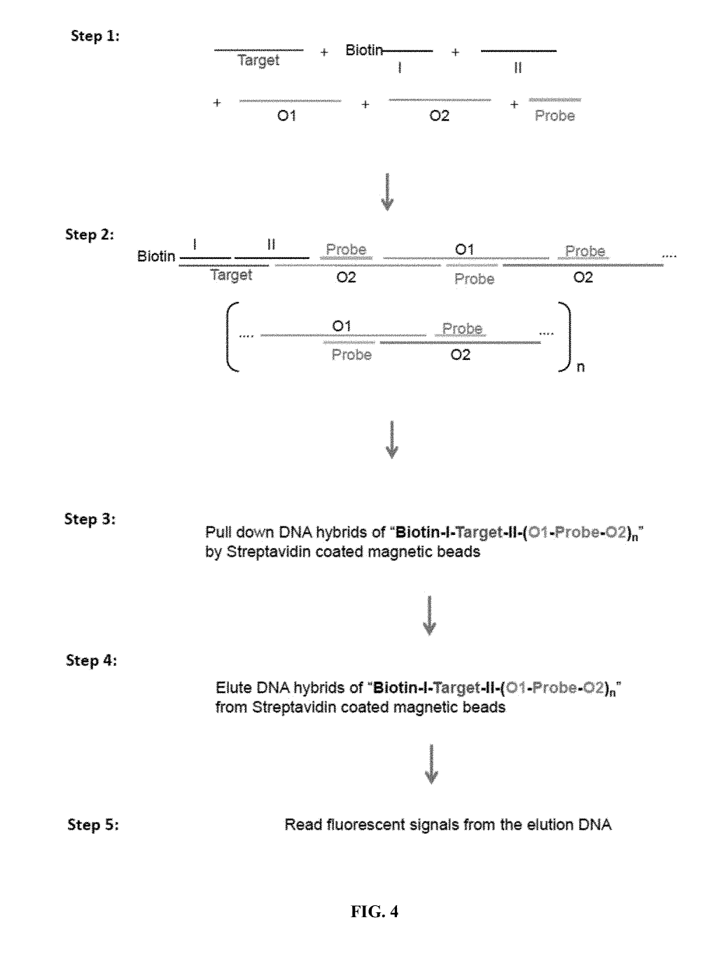

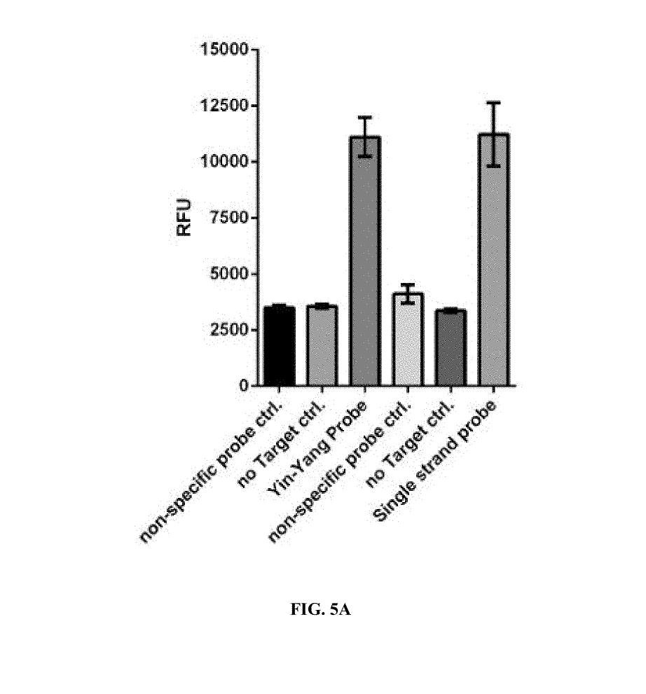

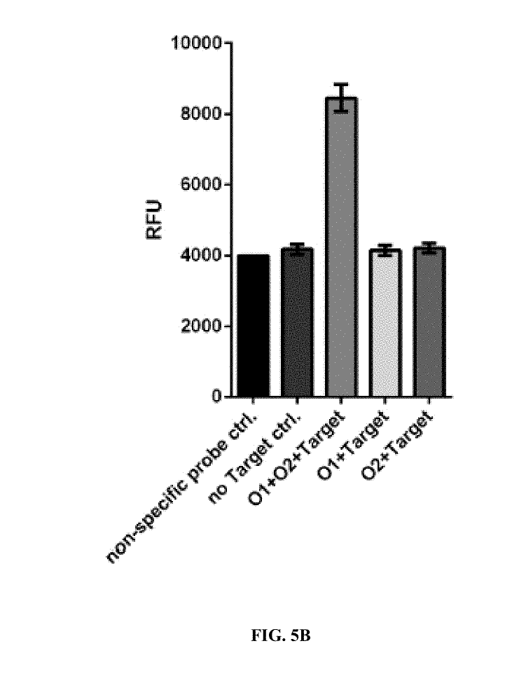

The result of the detection process is shown in FIGS. 5A and 5B. As shown in FIG. 5A, in the absence of the target nucleic acid, only background fluorescence was detected. In contrast, when the target nucleic acid was present, a strong fluorescence signal was detected. The detected fluorescence signal was specific to the target nucleic acid as confirmed by the result of using non-specific target probe, which gave rise to a signal comparable to the background. The result also showed that using single strand probe (P1+) generated a strong signal comparable to that using Yin-Yang probe. As shown in FIG. 5B, both the first and the second bridge probes are required for the formation of the probe composition as the presence of only one of the bridge probes gave rise to background signals.

### Example 2

This example illustrates the detection of a target nucleic acid using a probe composition includes a target probe, a capture probe, a bridge probe and a label probe.

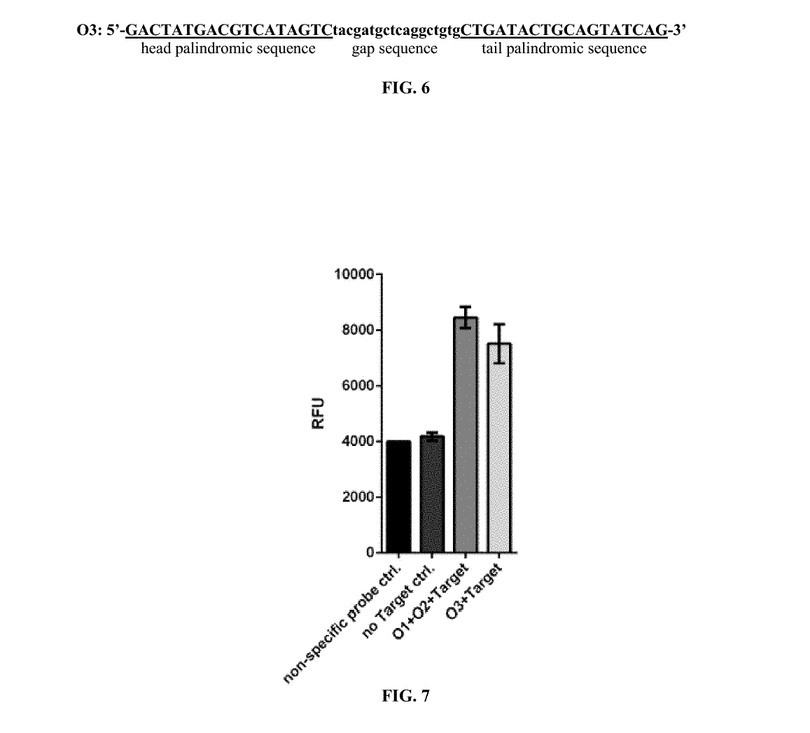

FIG. 6 shows the sequence of a bridge probe (O3), which contains palindromic sequences at the head and the tail region. The sequences of the target nucleic acid, the target probe, the capture probe and the label probe are the same as those used in Example 1.

The process of the detection was generally the same as Example 1.

FIG. 7 shows the detection result. As shown in FIG. 8, using O3 generates strong signal specific to the target nucleic acid.

### Example 3

This example illustrates the detection of a target nucleic acid using a probe composition without a pull-down step.

FIG. 8 shows the sequence of the target nucleic acid, the first bridge probe (H1), and the second bridge probe (H2). The label probe is the Yin-Yang probe P1 used in Example 1.

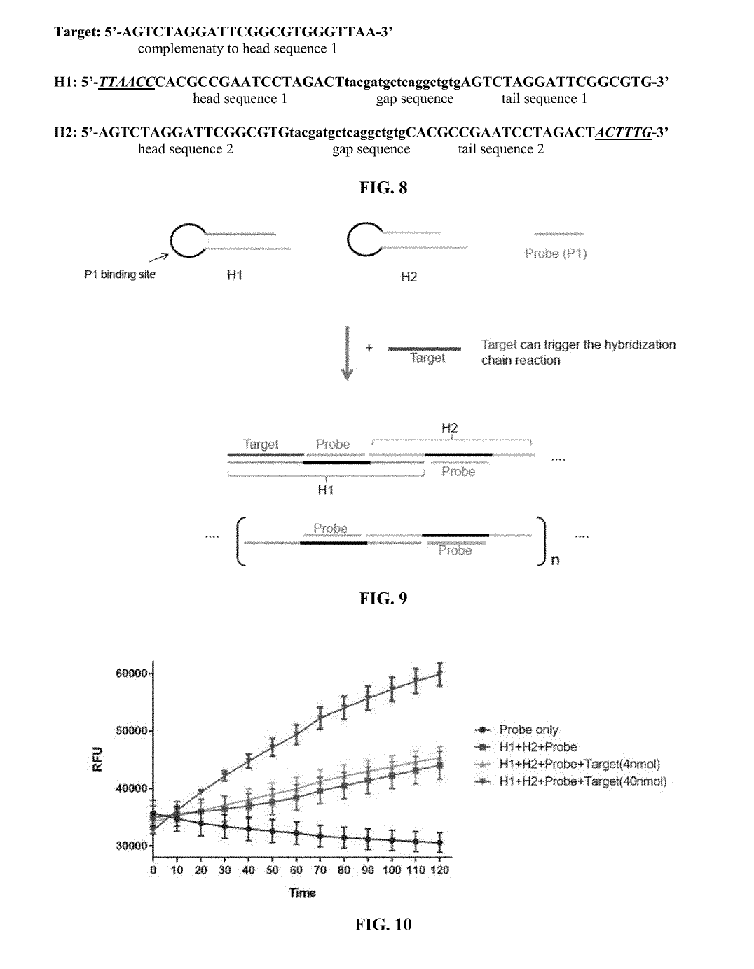

FIG. 9 illustrates the principle of the detection. In the first bridge probe, the head sequence is partially complementary to the tail sequence of the first bridge probe with six additional nucleotides at the 5′ end of the head sequence (underlined). The first bridge probe forms a hairpin structure in the absence of the target nucleic acid. In the second bridge probe, the head sequence is partially complementary to the tail sequence with six additional nucleotides at the 3′ end of the tail sequence (underlined). The head sequence of H1 is the same as the tail sequence of H2 except that the six additional nucleotides at the 5′ end of H1 is complementary to the six nucleotides at the 3′ end of H2. The tail sequence of H1 is the same as the head sequence of H2. The second bridge probe also forms a hairpin structure in the absence of the target nucleic acid.

In the presence of the target nucleic acid, which is complementary to the head sequence of H1, the hairpin structure of H1 is opened up, releasing the tail sequence of H1. The released tail sequence of H1 then hybridizes with the tail sequence of H2, which opens up the hairpin structure of H2 and releases the head sequence of H2. The label probes then hybridize to the gap sequences of H1 or H2. This results a probe composition through a chain reaction.

As shown in FIG. 10, when the label probes were mixed with H1 and H2 probes, fluorescent signals were detected even in the absence of the target nucleic acid but in a background level. However, adding the target nucleic acid (40 nmol) initiated the hybridization chain reaction and opened up the hairpin structures of H1 and H2 for the binding of the label probe, which resulted an elevated fluorescent signals.

What is claimed is:

1. A composition comprising:
a) a target probe capable of hybridizing to a target nucleic acid, said target probe comprising a pre-bridge region having a first tail nucleotide sequence;
b) at least one first bridge probe, comprising sequentially
(i) a first head region having a first head nucleotide sequence;
(ii) a first gap region having a gap nucleotide sequence;
(iii) a first tail region having the first tail nucleotide sequence;
c) at least one second bridge probe, comprising sequentially
(i) a second head region having a second head nucleotide sequence complementary to the first head nucleotide sequence;
(ii) a second gap region having the gap nucleotide sequence;
(iii) a second tail region having a second tail nucleotide sequence complementary to the first tail nucleotide sequence; and
d) a label probe capable of hybridizing to the first and the second gap nucleotide region, said label probe comprising a label.

2. The composition of claim 1, wherein the first head nucleotide sequence consists of 4-20 nucleotides.

3. The composition of claim 1, wherein the first tail nucleotide sequence consists of 4-20 nucleotides.

4. The composition of claim 1, wherein the gap nucleotide sequence consists of 6-20 nucleotides.

5. The composition of claim 1, wherein the target probe and/or the first bridge probe and/or the second bridge probe and/or the label probe comprises at least one nucleotide analog.

6. The composition of claim 5, wherein the nucleotide analog is a locked nucleic acid nucleotide.

7. The composition of claim 1, wherein the label is a fluorophore, a horse radish peroxidase or an alkaline phosphatase.

8. The composition of claim 1, wherein the target nucleic acid is selected from the group consisting of a DNA, a cDNA, a RNA, a mRNA, a rRNA, a miRNA, a Lnc RNA and a siRNA.

9. The composition of claim 1, wherein the target probe further comprises a label region having the gap nucleotide sequence.

10. The composition of claim 1, further comprising a capture probe capable of hybridizing to the target nucleic acid, said capture probe comprising a magnetic bead or a biotin.

11. The composition of claim 1, wherein the first bridge probe is the same as the second bridge probe.

12. A composition comprising:
a) a target probe capable of hybridizing to a target nucleic acid, said target probe comprising a pre-bridge region having a tail palindromic sequence;
b) at least one bridge probe, wherein the bridge probe comprises sequentially
(i) a first region having a head palindromic sequence;
(ii) a second region having a gap sequence;
(iii) a third region having the tail palindromic sequence; and
c) a label probe capable of hybridizing to the bridge probe, said label probe comprising a label.

13. A method of detecting a target nucleic acid in a sample, said method comprising:
a) contacting a target probe to the target nucleic acid, wherein the target probe is capable of hybridizing to the target nucleic acid and comprises a pre-bridge region having a tail nucleotide sequence;
b) associating a first bridge probe, a second bridge probe and a label probe with the target probe, wherein:
the first bridge probe comprises sequentially
(i) a first head region having a first head nucleotide sequence;
(ii) a first gap region having a gap nucleotide sequence; and
(iii) a first tail region having the tail nucleotide sequence;
the second bridge probe comprises sequentially
(i) a second head region having a second head nucleotide sequence complementary to the first head nucleotide sequence;
(ii) a second gap region having the gap nucleotide sequence;
(iii) a second tail region having a second tail nucleotide sequence complementary to the first tail nucleotide sequence;
the label probe capable of hybridizing to the bridge probe, said label probe comprising a label; and
c) detecting the presence, absence, or amount of the label probe associated with the target molecule.

14. The method of claim 13, wherein the first head nucleotide sequence consists of 4-20 nucleotides.

15. The method of claim 13, wherein the first tail nucleotide sequence consists of 4-20 nucleotides.

16. The method of claim 13, wherein the gap nucleotide sequence consists of 6-20 nucleotides.

17. The method of claim 13, wherein the label is a fluorophore, a horse radish peroxidase or an alkaline phosphatase.

18. The method of claim 13, wherein the target nucleic acid is selected from the group consisting of a DNA, a cDNA, a RNA, a mRNA, a rRNA, a miRNA, a Lnc RNA and a siRNA.

19. The method of claim 13, wherein the target probe further comprises the gap nucleotide sequence.

20. The method of claim 13, wherein the sample is selected from the group consisting of sera, plasma, saliva, urine, cell and tissue.

21. The method of claim 13, further comprising the step of capturing said target nucleic acid with a capture probe capable of hybridizing to the target nucleic acid, said capture probe comprising a magnetic bead or a biotin.

22. The method of claim 13, wherein the first bridge probe is the same as the second bridge probe.
